(19) United States
(12) Patent Application Publication
(10) Pub. No.: US 2004/0045045 A1
(43) Pub. Date: Mar. 4, 2004

(54) Animal model for toxicology and dose prediction

(76) Inventors: Jennie P. Mather, Millbrae, CA (US); Peter F. Young, Daly City, CA (US)

Correspondence Address: MORRISON & FOERSTER LLP, 755 PAGE MILL RD, PALO ALTO, CA, 94304-1018, US

(21) Appl. No.: 10/448,766

(22) Filed: May 30, 2003

Related U.S. Application Data
(60) Provisional application No. 60/384,715, filed on May 30, 2002.

(51) Int. Cl. A01K 67/027

(52) U.S. Cl. 800/14; 800/21

(57) ABSTRACT

The invention relates to the use of fetal tissues to generate a tissue model in a non-human animal. The tissue model comprises target tissues allowed to progress through development in vivo in a non-human host in order to obtain tissues having a mature phenotype that can be used to assess toxicity and/or efficacy of an agent.

### CROSS REFERENCE TO RELATED APPLICATIONS

This application claims the benefit of U.S. Provisional Application Serial No. 60/384,715, filed May 30, 2002, which is incorporated in its entirety by reference.

### FIELD OF THE INVENTION

The invention is in the field of cell biology. More specifically, it relates to the use of fetal tissues derived from one species and allowed to progress through development in vivo in a host of another species in order to obtain tissues having a mature phenotype that can be used to assess activity or toxicity of an agent.

### BACKGROUND OF THE INVENTION

The use of animal models is critical to the correct assessment of the efficacy and safety of new drugs. Tests performed on two species, usually rodents (rabbits, rats, mice, hamster, guinea pig etc.), and frequently primates, are required when filing an Investigational New Drug application (IND). The rodent models are less expensive but frequently suffer from problems of being evolutionarily too far removed from the human and therefore not adequately reflecting human physiology. This may be true for both efficacy and safety studies. While this has been a persistent problem for small molecule drugs, as more and more antibodies are moved into development, this problem becomes even more acute.

Antibodies recognize discrete sequences of amino acids and can be specific not only to a given protein but also to a specific species. For example an antibody to rat Epithelial Cell Adhesion Molecule (EpCAM) may recognize rat, but not mouse or human EpCAM (Stephan et al, Endocrinology 140:5841-5854,1999) and vice versa for anti-human EpCAM antibodies. This is true even though the protein sequence for the rat, mouse, and human EpCAMs is >98% conserved. It is to be expected that most antibodies for therapeutic use will have some degree of species specificity. Most will not react with rodent proteins, and some may be entirely specific for the human form of the protein, and not cross-react with non-human primate versions of the same protein.

Antibodies developed for the treatment of cancer are routinely tested for efficacy by injecting human tumor-derived cell lines subcutaneously into immunodeficient rats or mice (nu/nu or SCID). Since the animal's immune system does not attack the human cells, the human cells can grow into human tumors. The effect of the monoclonal antibodies on these tumors can then be studied by administering the human protein-specific monoclonal antibody to the mouse and the growth, shrinkage or death of the tumor measured. More rarely, tumor cells can be implanted under the kidney capsule, a well-vascularized area, and allowed to grow at this location. These are referred to as “Xenograft Models”.

These xenograft models, however, are not well adapted to performing drug safety assessment because the administered monoclonal antibodies will not bind to mouse or rat protein and therefore, could not harm the rodent host via an antibody-target mediated mechanism. Generally, safety studies must then be done in primates, if the monoclonal antibody is cross-reactive with primate cells, or await data from phase I human clinical trials. It would clearly be of great use to have a means to assess toxicity of these antibodies on normal post natal or adult human tissues at a stage earlier than clinical trial.

One known approach to assessing such safety is to use immunohistochemistry to determine the various human cell or tissue types that are bound by the antibodies. However, it is well known from antibody clinical trials that antibody binding alone is not predictive of safety. Some monoclonal antibodies bind to cancerous tissues but do not adversely affect their function, i.e., destroy the cancerous cells, or reduce proliferation of the cancerous cells. See, for example, Lewis et al Cancer Immunol Immunother 37:255-263 (1993); Herlyn et al J Immunol Methods 73:157-167 (1984); Fendly et al Cancer Research 50:1550-1558 (1990); and Balzer et al J Mol Med 77:699-712 (1999).

Furthermore, it is extremely difficult to obtain tissues of various tissue types from normal healthy adults and therefore, it is difficult to determine the effects of an antibody binding to normal tissue or to assess any toxicity of an antibody on normal tissues. Most studies using human tissue utilize tissues removed at autopsy. These tissues are variable in quality and disease status and frequently have undergone major changes. Alternatively, tissue can be obtained that is removed during surgery because it is adjacent to diseased tissue, such as cancer, and frozen or preserved immediately. This surgically removed tissue is more relevant to the living tissue than autopsy tissue, but because of its proximity to diseased tissue and/or treatments that the patient has undergone, the tissue may also be significantly different from normal tissue.

An animal model that would allow direct comparisons of the effect of an agent such as a monoclonal antibody, or other protein or small molecule drug, on human diseased (e.g., tumor) and normal tissues at the same time would be extremely valuable in assessing toxicity and efficacy of the agent. Animal models that would permit dose-ranging assessments of a therapeutic agent on a species other than the model animal would also be extremely valuable for purposes of designing the toxicology and other studies to be used to support the filing of an IND.

### SUMMARY OF THE INVENTION

The invention provides a non-human animal model of normal tissues having a mature phenotype and diseased tissues. This model is useful for, inter alia, determining the effects (e.g., toxicity) of various agents on normal and diseased tissues.

Accordingly, in one aspect, the invention is a method for generating a non-human vertebrate animal model having target normal tissue of mature phenotype and target diseased tissues both from a first vertebrate animal by: (a) implanting immature target normal tissue or normal tissue recombinants made from immature or progenitor cells of the first animal into a second, non-human vertebrate recipient animal at a location sufficient to support growth and maturation of said tissue; (b) allowing the target normal tissue of the first animal to develop into a tissue with a mature phenotype; (c) implanting target diseased tissue or diseased cells of the first animal into a non-human vertebrate recipient animal at a location sufficient to support growth of the diseased tissue; and (d) allowing the target diseased tissue or diseased cells from the first animal to grow.

In certain preferred embodiments, both the target normal tissue and the target diseased tissue from the first animal are implanted into different locations in one single non-human vertebrate animal. In other embodiments, the normal and the diseased target tissues are implanted in into different animals of the same species or of different species, depending on the comparative data desired. Preferably in this instance the implantings are made in parallel, or as close to contemporaneously as practicable, and more preferably on the same day. In some embodiments, both the implanted target normal tissue and the implanted target diseased tissue are human in origin.

Vertebrate animals suitable for having their tissue implanted into a second animal are many, and include by way of example and without limitation, mammals and other vertebrates, with particularly preferred species being mice, rats, birds, rabbits, cats, dogs, pigs, sheep, goats, deer, horses, cattle, humans, and non-human primates such as baboons, chimpanzees and monkeys.

The animal chosen to host or receive target tissue for implantation is preferably an immunodeficient non-human vertebrate animal. Animals suitable to serve this function are many, and include by way of example and without limitation, mammals and non-mammalian vertebrates. Particularly preferred embodiments are desirably selected from the group consisting of mice, rats, rabbits, frogs, birds, cats, dogs, pigs, sheep, goats, and non-human primates. In some embodiments, the non-human primate is baboon, chimpanzee, or monkey. Non-human vertebrate animals particularly preferred for receiving and hosting an implantation according to this invention are immunodeficient rodents (mouse and rat).

In another aspect, the invention is a tissue model for target normal tissue and/or diseased tissue from a first vertebrate animal species having mature phenotype present within an immunodeficient, second, non-human recipient or host animal, wherein the target normal and/or diseased first species tissue is selected from the group consisting of tissues of the following biological systems: Central Nervous System: Brain—Cerebrum (gray and white matter containing neurons, glia, etc.) and Brain—Cerebellum, Eye, Brainstem (pons, medulla, midbrain), Spinal Cord; Endocrine: Adrenal (cortex and medulla), Ovary, Pancreas (Islets of Langerhans and exocrine pancreas), Parathyroid, Pituitary (adenohypophysis and neurohypophysis), Testis, Thyroid (follicular epithelium, parafollicular cells, colloid, etc.); Breast: Breast (lobules, ducts, myoepithelial cells, etc.); Hematopoietic: Spleen, Tonsil, Thymus, Bone marrow (lymphocytes, monocytes/macrophages, granulocytes, erythroid precursors, megakaryocytes, mast cells, osteoclasts, osteoblasts), Peripheral blood cells (neutrophils, lymphocytes, monocytes, basophils, eosinophils, red blood cells, platelets); Respiratory: Lung (bronchi, bronchioles, alveoli, etc.); Cardiovascular: Heart, Blood vessels (arteries, veins, etc.); Gastrointestinal: Esophagus, Stomach (fundus), Small intestine (Ileum, jejunum or duodenum), Colon, Liver (portal triads, hepatic cells, etc.), Salivary Gland; Genitourinary: Kidney, Urinary, Bladder, Ureter, Urethra, Fallopian tube, Vagina, Placenta, Prostate, Uterus, Cervix; Musculoskeletal: Skeletal muscle; Skin: Skin (epidermis, appendages, dermis); Peripheral Nerve: Peripheral Nerve; Mesothelial cells: Lining cells from chest wall, abdominal wall, pericardium or from the surface of gastrointestinal, heart and/or lung samples, etc.

In certain particularly preferred aspects, the invention is an immunodeficient mouse or rat that has human tissue models for target normal human tissue having mature phenotype and diseased human tissue wherein the normal human tissue is selected from the group consisting of lung, prostate, kidney, pancreas, bladder, skin, liver, heart, colon, duodenum, stomach, thyroid, salivary gland, and thymus.

In another aspect, the invention is a method for assessing the effect of a treatment directed against a target diseased tissue by applying such treatment to an immunodeficient non-human vertebrate recipient animal of one species that has at least one each of a target normal tissue having mature phenotype and a target diseased tissue, wherein these target tissues are from vertebrate animal species different from the recipient animal, and assessing the effect of the treatment on the target normal and diseased tissues.

In certain aspects, the animal models of this invention are particularly useful for evaluating candidate treatments to be applied against a diseased tissue such as cancer, with the treatment candidates to be used for radio-, chemo-, or radiopharmaceutical therapy or radio-immunotherapy. The animal models of this invention are also useful for radio-imaging of neoplasms or tumors, and for the study of metastasis.

In other aspects, the invention is a method for determining a dose of an agent that is toxic to a target tissue by administering an agent to an immunodeficient recipient animal that has at least one of a target normal tissue having a mature phenotype and a target cancerous tissue from a donor animal, and assessing any toxic or deleterious or adverse effects of the agent on the normal and cancerous target tissues.

In another aspect, the invention is a method for identifying an agent that is toxic to target infected, diseased or cancerous cells to a greater extent than normal cells by administering an agent to an immunodeficient recipient animal that has both a target normal tissue having mature phenotype and an infected, diseased or cancerous target tissue from a donor animal, and identifying the agent that reduces the growth of or destroys the infected, diseased or cancerous target tissues to a greater extent than the normal tissues.

In another aspect, the invention is an method for determining an effective amount of an agent that is toxic to diseased or cancerous cells to a greater extent than normal cells by administering an agent to an immunodeficient recipient animal that has both a normal target tissue having mature phenotype and a cancerous human tissue from a donor animal and determining an amount of the agent that is effective on the normal and the diseased or cancerous target tissues.

In still other embodiments, whole animal-based screening assays are provided. In these embodiments, invention encompasses the use of non-human host animals that have already received target tissue implants according to the invention, or parts thereof, for testing the cytotoxic, cytostatic, antimicrobial, anti-inflammatory or other therapeutic properties of treatments administered to said animals, or for testing the activity of such treatments in controlling or inhibiting the development of cancer, infections and/or disease. Thus, according to another aspect of the invention, a method of screening and identifying or testing a treatment, drug or other substance or treatment for activity against the development of or in the treatment of cancer, infection and/or disease, is provided, comprising administering to a non-human host animal that has already received target tissue implants according to the teachings of this invention, with said treatment, drug or other substance concerned and detecting or noting any reduced incidence in the development of cancer, infection and/or disease, and reduction in morbidity, as compared with corresponding animals that did not receive the treatment, drug or substance, or detecting or noting an effectiveness in maintaining, restoring or improving bodily function.

### BRIEF DESCRIPTION OF THE DRAWINGS

FIG. 1 shows results when tissues from normal fetal organs that were placed in the kidney capsule (panels 1, 2, 3, 6, 7, and 8) or fat pad (panels 4 and 5) of nude (nu/nu) (panel 1,6,7,8) or SCID (panel 2,3,4,5) immune compromised mice and allowed to develop into more mature phenotype.

FIG. 2 shows the results from using matured tissues for safety/efficacy models. The kidneys of the animals are shown in the figure. The left side of FIG. 2 shows LnCAP tumors while the right side shows normal tissues. The upper panels are from treated animals while the lower panels are from control animals.

FIG. 3 shows immunohistochemistry of human prostate and human colon matured tissues from the experiment described in FIG. 2. Tissues were also stained with directly labeled mPA6 (anti-human EpCAM) antibody. Anti-human EpCAM antibody mPA6 does not bind to mouse EpCAM.

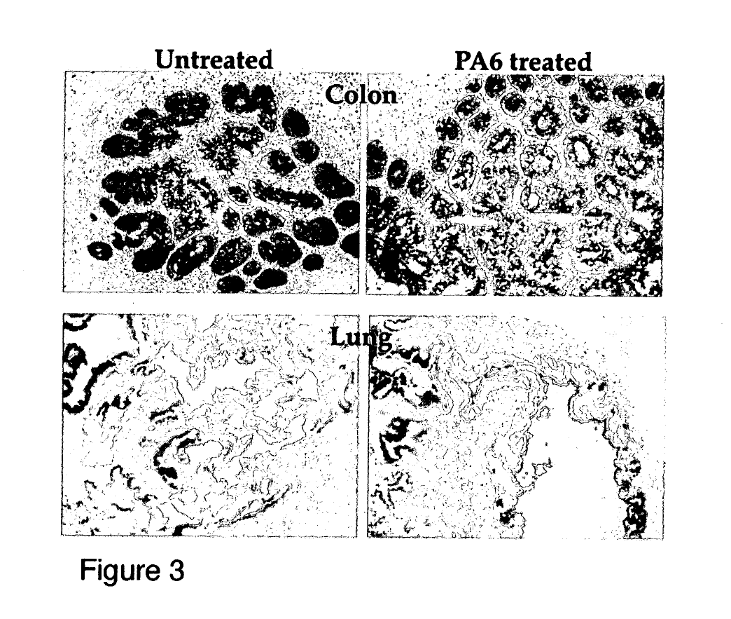

FIGS. 4A and 4B shows the results of a safety/efficacy study with mPA7 antibody. This antibody binds to the human PA7 antigen (CD46), which is present on normal prostate and pancreas epithelia and pancreatic cancer. This antibody does not recognize the mouse counterpart antigen.

FIG. 5 shows two different human tissue recombinants. A bladder epithelial progenitor cell line (hBLA) can be induced to form mature bladder epithelium (above) by combining with fetal bladder mesenchyme. However, the same cells will form mature prostate epithelium when combined with seminal vesicle mesenchyme (below). Arrows indicate positively staining cells.

FIG. 6 shows well-developed human colon, pancreas, heart and prostate tissues from human normal fetal organs that have been grown for six months in SCID mice.

FIG. 7 shows human and rat testis and liver mosaic tissues where the epithelial portion is derived from fetal progenitor cell lines and the stromal portion is derived from fetal rat mesenchyme. The tissues are recombined and allowed to develop for four to ten months to achieve a mature prototype.

### DETAILED DESCRIPTION OF THE INVENTION

We describe a non-human animal model in which target normal fetal tissues or tissue recombinants from human or other animal species, typically made using normal cell lines and dissected rat or mouse mesenchyme, are allowed to undergo developmental maturation in vivo. The resulting model can be used for assessing effects of an agent on both normal and/or diseased (e.g., cancerous) target tissue. This model is particularly advantageous as a human model, because normal mature human tissues representative of a variety of organs are not readily available for experimentation. The use of human fetal tissues or tissue recombinants made from human progenitor cells provides access to a wide variety of matured human tissues that is otherwise not readily available. For example, human pancreatic progenitor cells can give rise to human ductal, acinar, and islet cells. Using this non-human animal model (i.e., xenograft model), the effects of a therapeutic treatment regimen or agent can be readily assessed on all three types of mature human pancreatic cells.

Similarly, we describe non-human animal models in which the fetal tissue or tissue recombinants that are allowed to undergo developmental maturation in vivo are derived from other, non-human, vertebrate animals.

I. General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

II. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 2nd edition (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies of any isotype (IgA, IgG, IgE, IgD, or IgM), but also fragments thereof (such as Fab, Fab′, F(ab′)₂, Fv), single chain (ScFv), mutants thereof, fusion proteins comprising an antibody portion, chimeric antibodies (e.g., humanized antibodies), and any other modified configuration of the immunoglobulin molecule that comprises an antigen recognition site of the required specificity.

A “monoclonal antibody” refers to a homogeneous antibody population wherein the monoclonal antibody is comprised of amino acids (naturally occurring and non-naturally occurring) that are involved in the selective binding of an antigen. Monoclonal antibodies are highly specific, being directed against a single antigenic site. The term “monoclonal antibody” encompasses not only intact monoclonal antibodies and full-length monoclonal antibodies of any isotype (IgA, IgG, IgE, IgD, or IgM), but also fragments thereof (such as Fab, Fab′, F(ab′)₂, Fv), single chain (ScFv), mutants thereof, fusion proteins comprising an antibody portion, humanized monoclonal antibodies, chimeric monoclonal antibodies, and any other modified configuration of the immunoglobulin molecule that comprises an antigen recognition site of the required specificity and the ability to bind to an antigen. Such fragments and variants are well known in the art and are regularly employed both in vitro and in vivo. This invention is not intended to be limited as regards to the source of the antibody or the manner in which it is made (e.g., by hybridoma, phage selection, recombinant expression, transgenic animals, etc.). The fragments or analogues may be prepared using recombinant DNA methods or by synthetic methods such as solid-phase synthesis.

A “small molecule” refers to any composition of matter that is not made from amino acids and has a molecular weight of less than about 5000 daltons, preferably less than about 2500 daltons.

An “effective amount” or a “sufficient amount” of an antibody or other diagnostic or therapeutic treatment, substance or agent is an amount sufficient to effect beneficial or desired results, including the obtaining of diagnostic or prognostic information, clinical results such as shrinking the size of the tumor (in the cancer context, for example, breast or prostate cancer), retardation of diseased or cancerous cell growth, decreasing symptoms resulting from the disease, increasing the quality of life of those suffering from the disease, decreasing the dose of other medications required to treat the disease, delaying the progression of the disease, and/or prolonging survival of individuals. An effective amount can be administered in one or more administrations. For purposes of this invention, an effective amount of drug, compound, or pharmaceutical composition is an amount sufficient to reduce the proliferation of (or destroy) cancerous or diseased or infected cells and to reduce and/or delay the development, or growth, of metastases of cancerous cells, either directly or indirectly.

An “agent” is any element to which an individual can be exposed and can include, without limitation, antibodies, small molecules, proteins, pharmaceutical compounds (e.g., drugs), household chemicals, industrial chemicals, environmental chemicals, and other chemicals. The agents that may be tested in the animal models of this invention include but are not limited to immunochemotherapeutic agents, cytokines, chemotherapeutic agents and radiopharmaceuticals, and may also comprise internal or external radioactive agents as well as radiolabelled peptides. Gene therapy accomplished by methods well known in the art may also be evaluated using these models.

Many chemotherapeutic agents are known. Suitable agents for use in the practice of this invention may be selected from, but are not limited to, the following: allopurinol sodium, dolasetron mesylate, pamidronate disodium, etidronate, fluconazole, epoetin alfa, levamisole HCL, amifostine, granisetron HCL, leucovorin calcium, sargramostim, dronabinol, mesna, filgrastim, pilocarpine HCL, octreotide acetate, dexrazoxane, ondansetron HCL, ondansetron, busulfan, carboplatin, cisplatin, thiotepa, melphalan HCL, melphalan, cyclophosphamide, ifosfamide, chlorambucil, mechlorethamine HCL, carmustine, lomustine, polifeprosan 20 with carmustine implant, streptozocin, doxorubicin HCL, bleomycin sulfate, daunirubicin HCL, dactinomycin, daunorucbicin citrate, idarubicin HCL, plimycin, mitomycin, pentostatin, mitoxantrone, valrubicin, cytarabine, fludarabine phosphate, floxuridine, cladribine, methotrexate, mercaptipurine, thioguanine, capecitabine, methyltestosterone, nilutamide, testolactone, bicalutamide, flutamide, anastrozole, toremifene citrate, tamoxifen, estramustine phosphate sodium, ethinyl estradiol, estradiol, esterified estrogens, conjugated estrogens, leuprolide acetate, goserelin acetate, medroxyprogesterone acetate, megestrol acetate, levamisole HCL, aldesleukin, irinotecan HCL, dacarbazine, asparaginase, etoposide phosphate, gemcitabine HCL, trastuzumab, altretamine, topotecan HCL, hydroxyurea, interferon alfa-2b, mitotane, procarbazine HCL, vinorelbine tartrate, E. coli L-asparaginase, Erwinia L-asparaginase, vincristine sulfate, denileukin diftitox, aldesleukin, rituximab, interferon alfa-2a, paclitaxel, docetaxel, BCG live (intravesical), vinblastine sulfate, etoposide, tretinoin, teniposide, porfimer sodium, fluorouracil, betamethasone sodium phosphate and betamethasone acetate, letrozole, etoposide citrororum factor, folinic acid, calcium leucouorin, 5-fluorouricil, adriamycin, cytoxan, and diamino dichloro platinum.

In another aspect, the invention provides a method of evaluating the efficacy of a method of radioimaging of tumours or neoplasms, or of a method of treatment with a radio-labelled antibody, comprising the step of administering a radiolabelled, tumour-specific antibody to the animal model of the invention. The radiolabelled antibody may be a monoclonal or polyclonal antibody comprising a radiolabel, preferably selected from the group consisting of Technetium-99m, Indium-111, Iodine-131, Rhenium-186, Rhenium-188, Samarium-153, Lutetium-177, Copper-64, Scandium-47, Yttrium-90. Monoclonal antibodies labelled with therapeutic radionuclides such as Iodine-131, Rhenium-188, Holmium-166, Samarium-153 and Scandium-47, which do not compromise the immunoreactivity of antibodies and are not broken down in vivo, are especially preferred. The person skilled in the art will appreciate that other radioactive isotopes are known, and may be suitable for specific applications. The radioimaging may be conducted using Single Photon Emission Computer Tomography (SPECT), Position Emmission Tomography (PET), Computer Tomography (CT) or Magnetic Resonance Imaging (MRI). Correlative imaging, which permits greater anatomical definition of location of metastases located by radioimmunoimaging, is also contemplated.

An “individual” is a vertebrate, preferably a mammal, more preferably a human. Mammals include, but are not limited to, farm animals, sport animals, pets, primates, mice and rats.

As used herein, “immunodeficient” means an innate, acquired, or induced inability to develop a normal immune response. As described in more detail below, methods of reducing an individual's immune response level to below normal are well known in the art and their use in the context of this invention are within the ordinary skill of the practitioner.

As used herein, “treatment” is an approach for obtaining beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. As used herein, the term “treatment” includes prophylaxis. Treatment includes the cessation of growth of a tumor or other diseased tissue, the regression or disappearance of a detectable solid tumor or detectably infected or diseased tissue, or a prevention or diminution in metastasis of a tumor or the spread of infected or diseased tissue. “Palliating” a disease means that the extent and/or undesirable clinical manifestations of a disease state are lessened and/or the time course of the progression of the disease state is slowed or lengthened, as compared to a substance known to have little or no effect on these disease parameters.

As used herein, the models of this invention are valuable for the evaluation of a range of diseases and disorders, including by way of example and not in limitation: anemias, malignancies, autoimmune disorders, various immune dysfunctions and deficiencies, infection by pathogenic micro-organisms, diabetes, polycystic ovary disease, benign prostatic hypertrophy, osteoporosis, neurodegenerative diseases such as ALS, Alzheimer's disease, Parkinson's disease, muscular dystrophy, various metastatic and non-metastatic disorders such as various skin cancers, including melanoma; breast cancer; prostate cancer; renal cancer; liver cancer; lung cancer, brain cancer and other head and neck cancers, including glioblastomas; lymphomas, and leukemias, cardiovascular disease, renal impairment and disease, etc.

In certain aspects of this invention, the animal models facilitate the determination of a safe and effective dose of a therapeutic regimen that results in an individual's improved quality of life, as measured by a reduction in nausea, vomiting, loss of appetite, inability to sleep, decline in overall feeling, reduction in daily activity, fatigue and depression, without increasing other undesirable side effects.

In certain other aspects, the animal models of this invention are used to support the diagnostic and prognostic determinations of clinicians and researchers. This is accomplished by application of the methods of this invention to the use of patient tissue samples for implant, or for the use of substances derived from patient samples to treat an already-implanted tissue. The implanted tissue and tissue treatment methods are the same as described herein, and the methods of assessment of results are within the routine skill of the praticitioner.

III. Generating an Animal Model of Matured Tissue Phenotype

An animal model of mature tissue phenotype can be generated by several different methods. The recipient animal is preferably an immunodeficient animal. Animals that are not immunodeficient will mount an adverse immune response to the tissues from another species that are implanted in the animal, hence, the use of immunodeficient animals is highly encouraged. In one embodiment, the animal is a mouse or a rat. The immunodeficiency can be effected through genetic breeding (e.g., nu/nu, SCID, RAG, beige mice or nude rats, athymic mice or rats, etc.), genetic manipulation (e.g., genetic “knockout” technology), or by irradiating or chemically immunosuppressing the animals (e.g., treating with immunosuppressants such as cyclosporin or treating with radiation or another method that destroys the immune T and/or B cells).

The preferred animal subjects of this invention are vertebrate animals. The particularly preferred animal subject of the present invention is a mammal. By the term “mammal” is meant an individual belonging to the class Mammalia. The invention is particularly useful in the application to human tissue, although it is intended for veterinary uses as well.

The target human and other species tissues that are implanted into the recipient animal are immature in nature and can be derived from various sources. In one aspect, the human tissues are non-cancerous, non-diseased tissues, i.e., normal tissues, derived from human fetal tissues including, but not limited to, tissues of the following biological systems: Central Nervous System: Brain—Cerebrum (gray and white matter containing neurons, glia, etc.) and Brain—Cerebellum, Eye, Brainstem (pons, medulla, midbrain), Spinal Cord; Endocrine: Adrenal (cortex and medulla), Ovary, Pancreas (Islets of Langerhans and exocrine pancreas), Parathyroid, Pituitary (adenohypophysis and neurohypophysis), Testis, Thyroid (follicular epithelium, parafollicular cells, colloid, etc.); Breast: Breast (lobules, ducts, myoepithelial cells, etc.); Hematopoietic: Spleen, Tonsil, Thymus, Bone marrow (lymphocytes, monocytes/macrophages, granulocytes, erythroid precursors, megakaryocytes, mast cells, osteoclasts, osteoblasts), Peripheral blood cells (neutrophils, lymphocytes, monocytes, basophils, eosinophils, red blood cells, platelets); Respiratory: Lung (bronchi, bronchioles, alveoli, etc.); Cardiovascular: Heart, Blood vessels (arteries, veins, etc.); Gastrointestinal: Esophagus, Stomach (fundus), Small intestine (Ileum, jejunum or duodenum), Colon, Liver (portal triads, hepatic cells, etc.), Salivary Gland; Genitourinary: Kidney, Urinary, Bladder, Ureter, Urethra, Fallopian tube, Vagina, Placenta, Prostate, Uterus, Cervix; Musculoskeletal: Skeletal muscle; Skin: Skin (epidermis, appendages, dermis); Peripheral Nerve: Peripheral Nerve; Mesothelial cells: Lining cells from chest wall, abdominal wall, pericardium or from the surface of gastrointestinal, heart and/or lung samples, etc.

Particularly preferred tissue types for implantation are presently liver, lung, prostate, kidney, pancreas, heart, colon, duodenum, and thymus. The fetal tissue can be cut into small pieces sufficiently small to fit at the site of implantation but large enough to contain both stromal and epithelial elements of the tissue of origin. In one embodiment, the tissue is cut into dimensions of 10 mm×10 mm×10 mm. In another embodiment, the tissue is cut into dimensions of 5 mm×5 mm×5 mm. In another embodiment, the tissue is cut into dimensions of 1 mm×1 mm×1 mm. Several recombinants may be required to represent all different cell types from a large tissue containing many different cell types, such as the lung. Two or several pieces may be placed under the capsule of the same kidney.

In another embodiment, the tissue source is a human progenitor cell line derived from human fetal tissue, expanded, and recombined with rat or mouse mesenchyme selected to promote differentiation and maturation of the progenitor cells to one or more mature human cell types to form a human/rodent tissue recombinant. For example, such tissue recombinants can be human pancreatic progenitor cells (hPED) isolated and grown as described in U.S. Pat. No. 6,436,704, human Mullerian progenitor cells isolated and grown as described in U.S. Pat. No. 6,416,999, human ovarian progenitor cells isolated and grown as described in WO 01/77303 or human bladder progenitor cells (hBLA) as described in pending patent application PCT/US03/04547, the teaching of all of which are specifically incorporated by reference herein. Examples of other tissue-specific human progenitor cells or human cell lines that can be recombined with rat or mouse mesenchyme (for example) to form a tissue recombinant, according to the teachings of this invention, include, but are not limited to, those from ovary, bladder, pancreas, lung, skin, kidney, colon, thyroid, liver, heart, testis, and prostate. In another embodiment, the tissue source can be human mesenchyme derived cell lines. In yet another embodiment, the tissue source can be any human progenitor cell lines recombined with rodent mesenchymal tissue or appropriate human mesenchyme derived cell lines (e.g., pancreatic mesenchyme (hPEM) combined with human pancreatic progenitor cells such as hPED). In yet another embodiment, the human cell such as Schwann cells or neuroepithelial cells can be used for implantation into an immunodeficient animal.

In other embodiments, the tissues of the preceeding paragraph are derived alternatively from the progenitor cells or derived from fetal tissue or suitable cell lines of other non-human vertebrate species.

In yet another embodiment, the tissue source can be any human or other non-human vertebrate animal cell lines grown in a collagen matrix or other matrix material (e.g., plasma clot, EHS matrix, Matrigel, etc.). Each cell type is cultured in a medium designed to maintain the progenitor phenotype. Cells (1-3×10) are prepared and combined with the appropriate mesenchyme as described in U.S. Pat. No. 6,436,704 and U.S. Pat. No. 6,416,999.

In another aspect, the target tissues are infected, diseased and/or cancerous. For example, cell lines derived from human tumor or other diseased tissues can be used. These cells can be obtained from a biopsy or an autopsy, from transplantable tumors carried in immunodeficient mice or rats or from immortal cell lines established from human tumors or transformed in vitro.

Once normal and/or cancerous and/or infected and/or diseased tissue is obtained from a target animal, the target tissue is then implanted into the immunodeficient animal. Various sites of implantation are possible. In a preferred embodiment, the tissue or tissue recombinant is implanted under the kidney capsule of the immunodeficient animal. Use of nude mice for xenotransplantation generally of human tumors is known in the art. In other embodiments, the target tissue or tissue recombinant is implanted in the fat pad, subcutaneously, or any other location in the immunodeficient animal such that the target tissue or tissue recombinant can develop and mature and be located after a prolonged period of time (e.g., after a month or more). In another embodiment, tissues containing both epithelial and mesenchymal elements are trimmed to 1 mm cubed pieces and placed under the kidney capsule or into the fat pad of an immunodeficient animal.

Once the tissue source has been implanted into the immunodeficient animal, the tissue is allowed to develop for the amount of time required for maturation to the adult phenotype. This may differ from tissue to tissue but will be in the range of about 2 to 52 weeks, preferably about 4 to 36, more preferably about 6 to 24 weeks to reach the desired stage of development. The desired stage of development can be determined by implanting tissue from fetal (10-24 weeks of development) source and allowing development for a range of 6 to 24 weeks and looking at the histology and expression of specific, known markers for maturation in the resulting tissue. In general, according to the methods of this invention, more time is required for growth and maturation of normal tissues than for the growth of cancerous tissues.

In one embodiment, animals have 1-3 normal tissues that have been implanted under one kidney capsule and allowed to mature. Tumor cells can be implanted in the contralateral kidney capsule about 0-2 weeks prior to administration of one or more agent(s). The normal tissue can be allowed to mature in one animal and then that animal is euthanized and the tissue removed. The matured tissue can be split into two or more equivalent pieces and implanted in two or more recipient animals to generate animals that have matched pieces of normal human tissue. This is useful since one animal is the control and the other animal(s) is treated with one or more agent(s). The tumor cells can be implanted in the contralateral kidney capsule at this time.

In another embodiment, only normal tissues are implanted within the recipient animal. This is useful to test a treatment regimen or therapeutic agent, e.g., an antibody, to determine its effects on a variety of normal tissues. In some cases, the agent, e.g., an antibody, is known to have a deleterious effect on cancerous tissue. An animal model having only normal tissues can then be used to determine if the agent has any deleterious effects on other normal tissue over a range of doses.

II. Agents

The animal models described herein can be used to assess the effect of various agents including, but not limited to antibodies, small molecules, peptides, peptidomimetics, and proteins. Small molecules that can be used include synthetic chemical compounds, such as drugs being tested for FDA approval. Proteins that can be used include, but are not limited to, synthetic peptides and proteins, recombinant proteins, and naturally occurring proteins.

Various formulations of the therapeutic agents of this invention may be used for administration. In some embodiments, the agent may be administered undiluted. In other embodiments, the agent and a pharmaceutically acceptable excipient are administered, and may be in various formulations. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington: The Science and Practice of Pharmacy, 20th edition, Lippincott, Williams & Wilkins, Publishing.

Suitable formulations for parenteral administration include aqueous solutions of the active compounds in water-soluble form, for example, water-soluble salts. In addition, suspensions of the active compounds as appropriate for oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances that increase the viscosity of the suspension and include, for example, sodium carboxymethyl cellulose, sorbitol, and/or dextran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulation may be used simultaneously to achieve systemic administration of the active ingredient.

Suitable formulations for oral administration include hard or soft gelatin capsules, pills, tablets, including coated tablets, elixirs, suspensions, syrups or inhalations and controlled release forms thereof.

Various methods can be used to administer the agent to the animal model. In a preferred embodiment, the agent is administered intraperitoneally (i.p.). Other methods include, but are not limited to, oral, subcutaneous, intravenous, sub-capsular administration, intramuscular, or administration directly into the tissue or tumor. Administration may be enhanced by slow-release methodologies, including solid formulations such as a skin patch or pellet or encapsulated or coated dosage form, or if a liquid, through suitable liquid formulation or administration with an extra-corporeal or internally supported pumping mechanism.

The amount to be administered can be determined by various methods. In one embodiment, a dose of an agent, for example, an antibody, is determined by stepwise increments of the agent and the effects are monitored. In another embodiment, a skilled artisan uses an amount described in the art as a starting point for a dosage and stepwise increments above and below the reported amount are used to determine effects. In another embodiment, a dosage is used that reflects the physiological amount that an individual (e.g., a human) would experience if undergoing a treatment protocol or in routine daily exposure.

Generally, these agents are formulated for administration by injection (e.g., intraperitoneally, intravenously, subcutaneously, intramuscularly, etc.), although other forms of administration (e.g., oral, mucosal, etc) can be also used. Accordingly, the therapeutic agents of this invention are preferably combined with pharmaceutically acceptable excipients such as saline, Ringer's solution, dextrose solution, and the like. The particular dosage regimen, i.e., dose, timing and repetition, will depend on the particular individual and that individual's medical history. Generally, any of the following doses may be used: a dose of at least about 50 mg/kg body weight; at least about 10 mg/kg body weight; at least about 3 mg/kg body weight; at least about 1 mg/kg body weight at least about 750 microg/kg body weight; at least about 500 microg/kg body weight; at least about 250 microg/kg body weight; at least about 100 microg/kg body weight; at least about 50 microg/kg body weight; at least about 10 microg/kg body weight; at least about 1 microg/kg body weight, or more, is administered. Empirical considerations, such as the half life, generally will contribute to determination of the dosage. Agents that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is based on reducing the number of cancerous cells, maintaining the reduction of cancerous cells, reducing the proliferation of cancerous cells, or delaying the development of metastasis. Alternatively, sustained continuous release formulations of the agents of this invention may be appropriate. Various formulations and devices for achieving sustained release are known in the art.

In one embodiment, dosages for therapeutic agent may be determined empirically in individuals who have been given one or more administration(s). Individuals are given incremental dosages of therapeutic agent. To assess efficacy of the therapeutic agent, the specific cancer disease state can be followed by methods such as direct measurement of tumor size via palpation or visual observation, indirect measurement of tumor size by x-ray or other imaging techniques, an improvement as assessed by direct tumor biopsy and microscopic examination of the tumor sample, the measurement of an indirect tumor marker (e.g., PSA for prostate cancer), a decrease in pain, paralysis, impairment of speech, vision, breathing or other disability associated with the tumor, increased appetite, or an increase in quality of life as measured by accepted tests or prolongation of survival. It will be apparent to one of skill in the art that the dosage will vary depending on the individual, the type of cancer, the stage of cancer, whether the cancer has begun to metastasize to other location in the individual, and the past and concurrent treatments being used.

Other formulations include suitable delivery forms known in the art including, but not limited to, carriers such as liposomes. See, for example, Mahato et al. (1997) Pharm. Res. 14:853-859. Liposomal preparations include, but are not limited to, cytofectins, multilamellar vesicles and unilamellar vesicles.

In some embodiments, more than one therapeutic agent may be present. Such compositions may contain one or more than one therapeutic agent (may contain at least one, at least two, at least three, at least four, at least five different therapeutic agents) that is reactive against, for example, ovarian, lung, prostate, pancreatic, colon, or breast cancer cells. A mixture of therapeutic agents, as they are often denoted in the art, may be particularly useful in treating a broader range of population of individuals.

Assessment of disease is performed using standard methods in the arts, such as imaging methods and monitoring appropriate marker(s), as discussed in more detail below.

The timing of the administration of the agent will depend on the nature of the agent. In one embodiment, the agent is an antibody that is administered at an effective amount to reduce growth of cancerous cells, cancerous tissues, or tumors. One of skill in the art may determine the efficacy of multiple administrations at low concentrations versus a single administration at a higher concentration without undue experimentation.

The agent may be administered once or on a schedule requiring 1-7 injections or more per week over a period of weeks. The dosage, administration schedule, and duration will generally reflect that shown to be efficacious in treating the infection or disease.

III. Assessment of Efficacy/Toxicity Model

The animal models containing the tissue implant or tissue recombinant is allowed to develop into partially or completely adult normal matured tissue phenotype. To generate an animal model for assessing effects of an agent (e.g., toxicity), these animals containing matured target tissues under one kidney capsule can then be implanted with target diseased, infected or cancerous cells under the opposite kidney capsule (or other site) and treated with the agent (e.g., monoclonal antibody or other drug). After treatment the animal is sacrificed and the human diseased, infected or cancerous and normal tissue xenografts are removed, and analyzed to assess the effect of the agent. A skilled artisan can determine effects of the agent on both normal and diseased, infected or cancerous target tissue by monitoring the gross morphology, cellular morphology, the amount of necrosis or apoptosis, size of diseased, infected or cancerous tissue and/or function (e.g., insulin secretion for pancreatic tissue) or presence or absence of known markers of normal and abnormal cell functions. For example, staining with Ki67 antibody can be used to visualize the number of dividing cells in a tissue (Grogan et al. Blood 75:2714-9,1988). For therapeutic antibodies, the animal model can be used to identify an antibody and the dosage of an antibody that is effective in killing cancerous tissue or reducing the size of the tumor while having little or no effect on the corresponding normal tissue or other normal tissues expressing the antigen bound by the antibody.

Methods for assessing the effect of a therapeutic treatment regime on an individual will vary depending on the treated condition and the method of treatment, and a range of such methods are well known in the art. By way of illustration and not limitation, such methods include those discussed above, and also methods for assessing hypertrophy, hyperplasia, apoptic death, differential protein or steroid secretion, metabolic activity, and morphology changes.

Matured tissue can also be used to assess efficacy and toxicity of small molecule drugs by systemic treatment of the animal with agent then measuring a function of the target tissue, e.g., human insulin production by “matured” human pancreas. Another use of this animal model is an efficacy model whereby animals with different matured tissues (both normal and diseased) in each kidney are used to assess the effects of long term (e.g., days to months) treatment with the agent.

### EXAMPLES

### Example 1

Generation of Non-Human Animal Models

Tissues from normal fetal organs (colon, heart, kidney, liver, lung, ovary, and oviduct) were trimmed to 1 mm cubed pieces and placed in the kidney capsule or fat pad of nude (nu/nu) or SCID immunocompromised mice. The tissues were left in the animals for 6-40 weeks to allow time for the development into mature tissues. The animal was euthanized and the tissues were removed and sectioned for H&E staining and immunohistochemical evaluation.

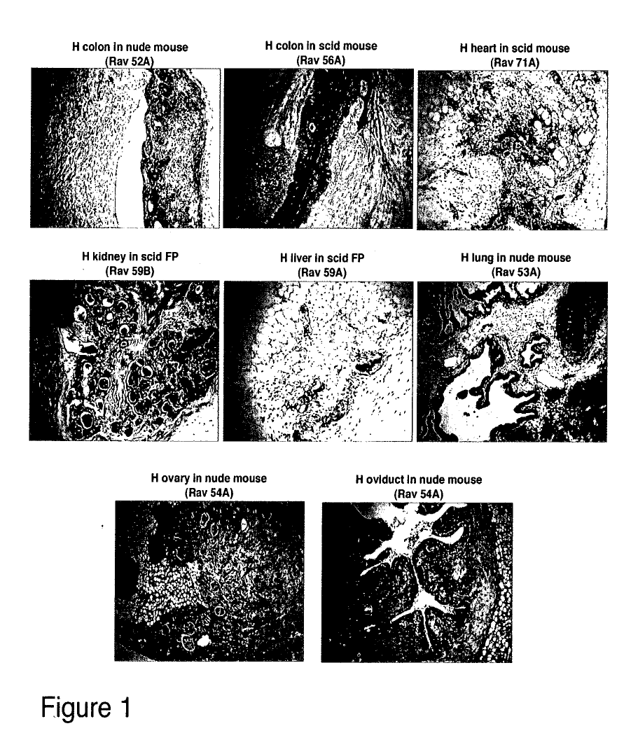

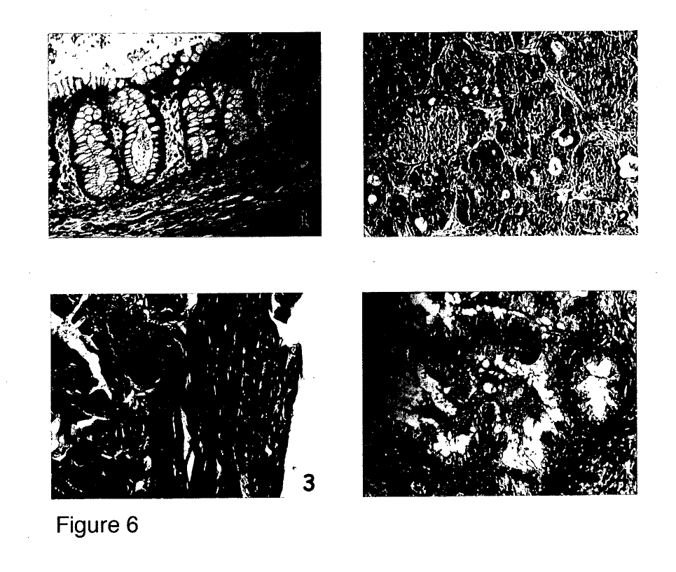

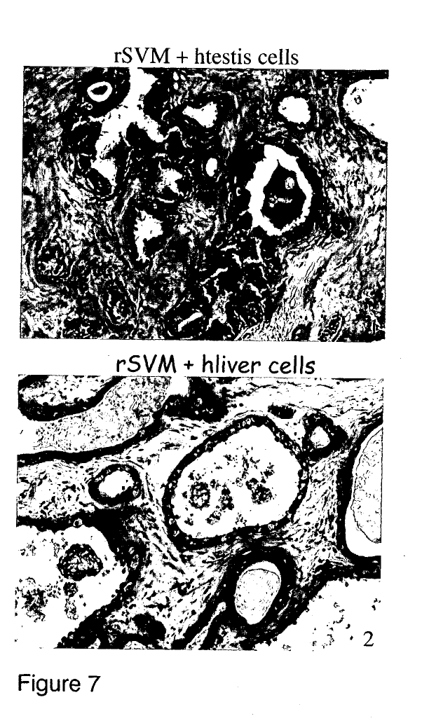

FIG. 1 shows the results of one series of implantations where the tissues were allowed to mature for 4 months. In this example, all references to “Panels” refer to FIG. 1. Panels 1, 2, 3, 6, 7, and 8 show implantation under the kidney capsule while panels 4 and 5 show implantation under the fat pad. Panels 1, 6, 7, and 8 show implantation of normal fetal organs in a nude (nu/nu) mouse while Panels 2, 3, 4, and 5 show implantation of normal fetal organs in a SCID mouse. Kidney, heart and liver tissue fail to develop when placed in the kidney capsule (Panel 3). While kidney tissue does not develop and mature when placed in the kidney capsule (not shown), it will develop in the fat pad (Panel 4). While the 1 mm cubed piece of heart did not develop in this experiment (Panel 5), in another set of experiments, long, thin (at 0.6×2 mm) pieces of heart tissue, containing cells from both the auricle and the ventricle, did survive and develop both fat and muscle tissue when implanted under the kidney capsule for 7 months. FIG. 6 shows well-developed colon, pancreas, heart, and prostate after six months in the host mouse. The dissected fetal liver tissue did not develop at either the fat pad or the kidney capsule implantation sites (Panel 5). However, tissue recombinants using human liver epithelial progenitors and rat fetal seminal vesicle mesenchyme (rSVM) did differentiate into structures with a well-defined architecture. Testis epithelial progenitor cells combined with rSVM developed duct-like tubular structures similar to germinal cell deficient testis since the testicular germline stem cells are not present in the original cultures (see FIG. 7) Oviduct development is extensive (Panel 8) while lung and ovary (Panels 6 and 7) mature but do not have the same structural development as the tissues in vivo. However, lung tissue that had been allowed to develop in vivo (kidney capsule) for 7 months did develop adult cell morphologies including ciliated epithelial cells. Since immune deficient mice have a shorter than normal lifespan, these long development times may require transplanting the piece of normal tissue to a younger (e.g., 6-10 week) animal after 5 to 6 months of development.

### Example 2

Use of Matured Tissues for Safety/Efficacy Models

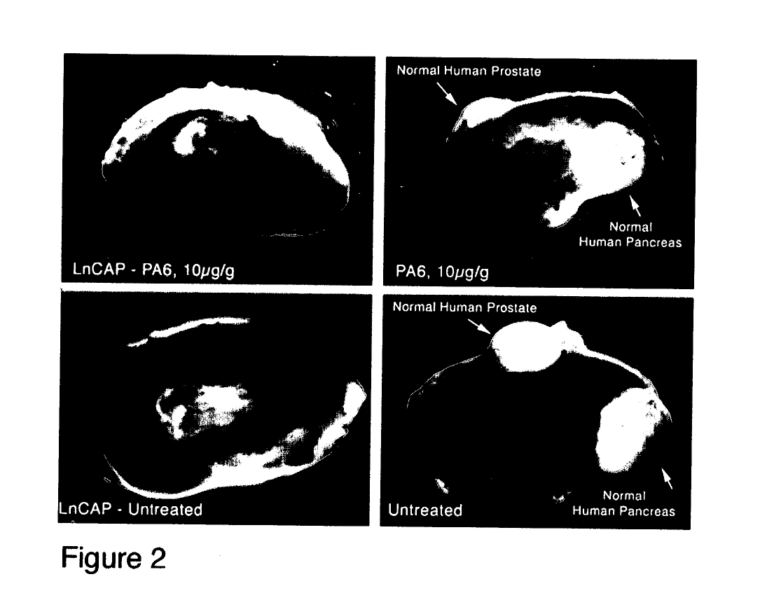

Normal human prostate and pancreas pieces were placed under the kidney capsule and allowed to mature for 6 weeks. At this time, human prostate cancer cells (LnCAP) were placed under the contralateral kidney capsules of the same animals and allowed to grow for one additional week. At day 7 after implanting the LnCAP tumors, one animal was treated with 10 ugm/gm PA6 antibody (anti-human EpCAM) by i.p. injection. The control animal was treated with saline injections. 4 injections were given over a two week period. At the end of this time, the animals were euthanized and the tumor and normal tissue xenografts examined. The kidneys of the animals are shown in FIG. 2. The left side of FIG. 2 shows LnCAP tumors while the right side shows normal tissues (9 weeks total in the animal). The upper panels are from treated animal while the lower panels are from control animals. Additional treated animals contained normal colon tissue.

### Example 3

Immunohistochemistry of Human Prostate and Human Colon Matured Tissues

Immunohistochemistry of human prostate and human colon matured tissues from the experiment described in FIG. 2. Although the tumor was impacted by the antibody treatment with cell death and hemorrhaging, the normal tissues were unaffected by the antibody (A-D). In order to determine whether the tissues contained the antibody target (EpCAM), tissues were stained with directly labeled PA6 (anti-human EPCAM) antibody. The tissues, both treated and untreated show binding of the antibody. The matured human prostate tissue also stained strongly for prostate specific antigen (PSA), a marker for prostate cells.

### Example 4

Safety/Efficacy Study on mPA7 Antibody

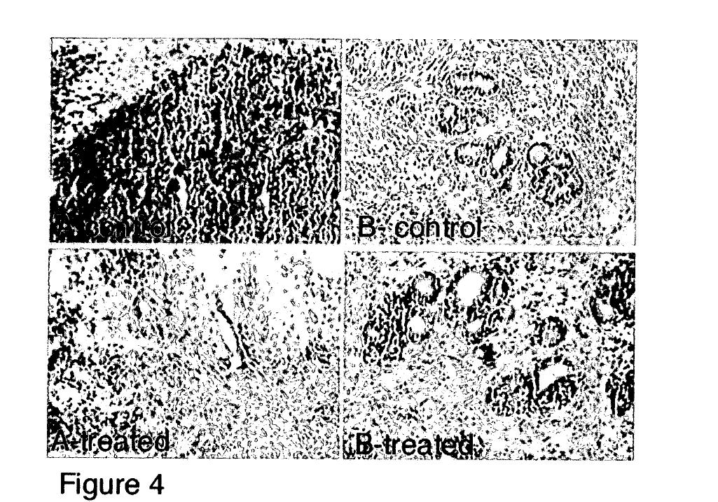

A similar experiment was performed with human fetal pancreas and prostate tissue. The pancreas and prostate tissue was allowed to mature for 11 weeks before implantation of LnCAP prostate tumor tissue in the contra-lateral side. The animals were treated as in Example 2, with 50 ug/gm×4 doses of mPA7 antibody. As seen in FIG. 4A the antibody treatment caused the tumor tissue to disappear, leaving only scar tissue. The normal tissues shown in the H&E stained sections in 4B were unaffected by the antibody treatment.

### Example 5

Normal Tissue Recombinants Developed from Human Progenitor Cells and Rat Fetal Mesenchyme

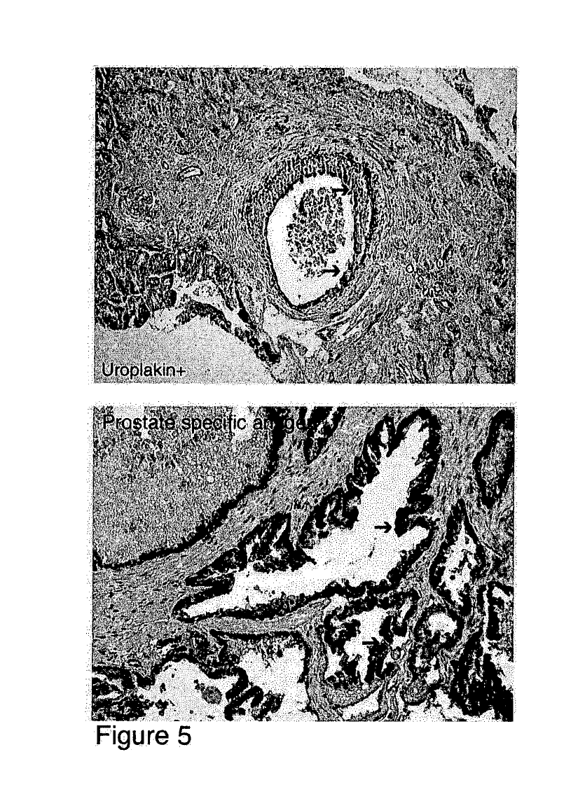

An alternative to using whole pieces of fetal tissue to mature to adult phenotype is to use human progenitor cell lines recombined with rodent mesenchymne to derive a tissue in which a portion of the cells, those derived from the progenitor cell line, are of an adult human phenotype. An example of this is shown in FIG. 5. Here the hBLA (human bladder epithelial progenitor) cell line was recombined with rat fetal bladder mesenchyme to form a tissue mosaic containing rat mesenchyme and human epithelial cells with an adult bladder epithelial phenotype. This tissue is shown in the upper panel stained for uroplakin, a marker for human bladder umbrella cells. The same cells could be recombined with rat fetal seminal vesicle mesenchyme and allowed to mature for 6 months in vivo to form a tissue mosaic with rat mesenchyme and human adult prostate epithelium (stained for human prostate specific antigen (PSA) in the lower panel). Similarly, tissue recombinants have been made using human fetal liver cells, human fetal pancreatic cells (see patent U.S. Pat. No. 6,436,704), or human uterine/vaginal/fallopian tube progenitor cells (see patent U.S. Pat. No. 6,416,999), recombined with appropriate rat mesenchyme to make human/rat mosaic tissues.

It is understood that the examples and embodiments described herein are for illustrative purposes only and that various modifications or changes in light thereof will be suggested to persons skilled in the art and are to be included within the spirit and purview of this application and the scope of the appended claims. All publications, patents and patent applications cited herein are hereby incorporated by reference in their entirety for all purposes to the same extent as if each individual publication, patent or patent application were specifically and individually indicated to be so incorporated by reference.

We claim:

1. A method for generating a non-human vertebrate animal model having target normal tissue of mature phenotype and target diseased tissues both from a first vertebrate animal, comprising the steps of:
(a) implanting immature target normal tissue or normal tissue recombinants made from immature or progenitor cells of the first animal into a second, non-human vertebrate recipient animal at a location sufficient to support growth and maturation of said tissue;
(b) allowing the target normal tissue of the first animal to develop into a tissue with a mature phenotype;
(c) implanting target diseased tissue or diseased cells of the first animal into a non-human vertebrate recipient animal at a location sufficient to support growth of the diseased tissue; and
(d) allowing the target diseased tissue or diseased cells from the first animal to grow.

2. The method of claim 1, wherein both the target normal tissue and the target diseased tissue from the first animal are implanted into different locations in one single non-human vertebrate animal.

3. The method of claim 1, wherein the normal and the diseased target tissues are implanted into separate animals.

4. The method of claim 1, wherein the donor and the normal and the recipient animals are of different species.

5. The method of claim 1, wherein both the implanted target normal tissue and the implanted target diseased tissue are human in origin.

6. The method of claim 1, wherein the implanted target tissue is derived from a species selected from the following group: mice, rats, rabbits, birds, cats, dogs, pigs, sheep, goats, deer, horses, cattle, humans, and non-human primates.

7. The method of claim 6, wherein the non-human primate is a baboon, chimpanzee or monkey.

8. The method of claim 1, wherein the non-human vertebrate recipient animal is selected from the group consisting of immunodeficient mice, rats, rabbits, cats, frogs, birds, dogs, pigs, sheep, goats, and non-human primates.

9. The method of claim 8, wherein the animal is an immunodeficient rodents.

10. A tissue model for the evaluation of target tissue from a first vertebrate animal species having mature phenotype present within an immunodeficient, second, non-human recipient or host animal, wherein the target normal and/or diseased first species tissue is selected from the group consisting of tissues of the following biological systems: Central Nervous System: Brain—Cerebrum (gray and white matter containing neurons, glia, etc.) and Brain—Cerebellum, Eye, Brainstem (pons, medulla, midbrain), Spinal Cord; Endocrine: Adrenal (cortex and medulla), Ovary, Pancreas (Islets of Langerhans and exocrine pancreas), Parathyroid, Pituitary (adenohypophysis and neurohypophysis), Testis, Thyroid (follicular epithelium, parafollicular cells, colloid, etc.); Breast: Breast (lobules, ducts, myoepithelial cells, etc.); Hematopoietic: Spleen, Tonsil, Thymus, Bone marrow (lymphocytes, monocytes/macrophages, granulocytes, erythroid precursors, megakaryocytes, mast cells, osteoclasts, osteoblasts), Peripheral blood cells (neutrophils, lymphocytes, monocytes, basophils, eosinophils, red blood cells, platelets); Respiratory: Lung (bronchi, bronchioles, alveoli, etc.); Cardiovascular: Heart, Blood vessels (arteries, veins, etc.); Gastrointestinal: Esophagus, Stomach (fundus), Small intestine (Ileum, jejunum or duodenum), Colon, Liver (portal triads, hepatic cells, etc.), Salivary Gland; Genitourinary: Kidney, Urinary, Bladder, Ureter, Urethra, Fallopian tube, Vagina, Placenta, Prostate, Uterus, Cervix; Musculoskeletal: Skeletal muscle; Skin: Skin (epidermis, appendages, dermis); Peripheral Nerve: Peripheral Nerve; Mesothelial cells: Lining cells from chest wall, abdominal wall, pericardium or from the surface of gastrointestinal, heart and/or lung samples, etc.

11. An immunodeficient rodent having human tissue models for target normal human tissue having mature phenotype and diseased human tissue wherein the normal human tissue is selected from the group consisting of lung, prostate, kidney, pancreas, bladder, skin, liver, heart, colon, duodenum, stomach, thyroid, salivary gland, and thymus.

12. A method for assessing the effect of a therapeutic treatment directed against a target diseased tissue, comprising the steps of
(a) applying such treatment to an immunodeficient non-human vertebrate recipient animal of one species that has at least one each of a target normal tissue having mature phenotype and a target diseased tissue from another vertebrate animal species, and
(b) assessing the effect of the treatment on the target normal and diseased tissues.

13. The method of claim 12, wherein the therapeutic treatment is selected from the group consisting of radio-, chemo-, or radiopharmaceutical therapy or radio-immunotherapy.

14. The method of claim 12, wherein the therapeutic treatment comprises the administration of agents useful for radio-imaging of tumors.

15. A method for determining a dose of an agent that is toxic to a target tissue, comprising the steps of:
(a) administering an agent to an immunodeficient recipient animal that has at least one of a target normal tissue having a mature phenotype and a target cancerous tissue from a donor animal, and
(b) assessing for any toxic effects of the agent on the normal and cancerous target tissues.

16. A method for identifying an agent that is toxic to target diseased or cancerous cells to a greater extent than normal cells, comprising the steps of:
(a) administering an agent to an immunodeficient recipient animal that has a target normal tissue having mature phenotype and a diseased or cancerous target tissue from a donor animal, and
(b) identifying the agent that reduces the growth of or destroys the diseased or cancerous target tissues to a greater extent than normal tissues.

17. A method for determining an effective amount of an agent that is toxic to diseased or cancerous cells to a greater extent than normal cells, comprising the steps of:
(a) administering an agent to an immunodeficient recipient animal that has a normal target tissue having mature phenotype and a cancerous human tissue from a donor animal, and
(b) determining an amount of the agent that is effective on the normal and diseased or cancerous target tissues.

18. A whole animal-based screening assay, comprising the use of nonhuman host animals that have already received target tissue implants according to the invention, or parts thereof, for testing the cytotoxic, cytostatic, antimicrobial, anti-inflammatory or other therapeutic properties of treatments administered to said animals, or for testing the activity of such treatments in controlling or inhibiting the development of cancer, infections and/or disease.

19. A method of screening and identifying or testing a treatment, drug or other substance for activity against the development of or in the treatment of cancer, infection and/or disease, comprising the steps of
(a) treating a non-human host animal that has already receive target tissue implants according to the teachings of this invention, or a part thereof, with said treatment, drug or other substance concerned, and
(b) detecting or noting any reduced incidence in the development of cancer, infection and/or disease, and reduction in morbidity, as compared with corresponding animals that are not treated with the treatment, drug or substance, or detecting or noting an effectiveness in maintaining, restoring or improving bodily function.
